(19) United States
(12) Patent Application Publication
(10) Pub. No.: US 2013/0005679 A1
(43) Pub. Date: Jan. 3, 2013

(54) Multivitamin Compositions and Methods for Improving Health

(76) Inventor: Tasneem Bhatia, Atlanta, GA (US)

(21) Appl. No.: 13/342,931

(22) Filed: Jan. 3, 2012

Related U.S. Application Data
(60) Provisional application No. 61/429,220, filed on Jan. 3, 2011.

(51) Int. Cl. A61K 31/714; A61P 15/00; A61P 25/20; A61P 3/02

(52) U.S. Cl. 514/52

(57) ABSTRACT

Provided are multivitamin compositions comprising vitamin B1, vitamin B2, vitamin B5, vitamin B6, vitamin B12, folic acid, magnesium glycinate, and selenium. These compositions are effective and natural treatments for improving health in a mammal. In some embodiments, the multivitamin compositions are administered to a female mammal to normalize a menstrual cycle. In other embodiments, the multivitamin compositions are administered to treat a sleep disorder.

### CROSS-REFERENCE TO RELATED APPLICATIONS

This application claims the priority benefit of U.S. Provisional Patent Application No. 61/429,220 filed Jan. 3, 2011.

### FIELD OF THE INVENTION

This invention relates to multivitamin compositions and methods for improving health.

### BACKGROUND OF THE INVENTION

There are eight different types of B vitamins: vitamin B1 (thiamine), vitamin B2 (riboflavin), vitamin B3 (niacin) vitamin B5 (pantothenic acid), vitamin B6, (pyridoxine), vitamin B7 (biotin), vitamin B9 (folic acid), and vitamin B12 (cyanocobalamin). B vitamins are essential for many aspects of the body's functioning, but they have been closely tied to both hormone metabolism and neurologic development and function. Accordingly, a vitamin B deficiency can have a serious impact on overall health.

Deficiencies in vitamin B1 correlate with a neurological disorders such as Wernicke syndrome and Korsakoff syndrome wherein neurologic function is impaired. Vitamin B1 deficiency is also associated with muscle weakness and the nutritional deficiency disease Beriberi. Vitamin B2 is associated with mitochondrial function and has been used as a treatment for autistic patients. Niacin, or vitamin B3, has been found to reduce atherosclerosis, boost levels of HDL (good) cholesterol and lower triglycerides. Scientists at the International Agency for Research on Cancer in Lyon, France have also found that vitamin B6 and methionine are strongly associated with reducing lung cancer risk in people who have never smoked. Biotin plays a key role in the health of the skin, nerves, digestive tract, and metabolism. Biotin supplements have been used successfully with chromium to treat type 2 diabetes. Folic acid is recommended as a supplement for all pregnant women as it helps to prevent neural tube defects such as spina bifida. Folic acid deficiency can also cause anemia, as can a vitamin B12 deficiency. Researchers at the Jean Mayer USDA Human Nutrition Center on Aging at Tufts University focus many of their studies on the effects of vitamin B6, vitamin B12 and folic acid. These researchers have found that metabolic impairments induced by a diet deficient in folic acid, vitamin B12 and vitamin B6 resulted in cognitive dysfunction and reductions in brain capillary length and density in a mouse model and may serve as evidence of the role of B vitamin deficiency in Alzheimer's disease.

Given the importance of B vitamins and their role in preventing neurologic and other disorders, there remains a need for multivitamin supplements that contain an effective array of B vitamins.

### SUMMARY OF THE INVENTION

The present invention answers the need for B complex multivitamin compositions that can be used to improve health. In some embodiments, the multivitamin compositions of the present invention are used to improve neurologic health. These compositions comprise vitamin B1, vitamin B2, vitamin B5, vitamin B6, vitamin B12, folic acid, magnesium glycinate, and selenium. In a preferred embodiment, the multivitamin composition comprises a combination of vitamin B1, vitamin B2, vitamin B5, vitamin B6, vitamin B12, folic acid, magnesium glycinate and selenium wherein the amounts of one or more of those components is as follows: about 25 mg of vitamin B1, about 25 mg of vitamin B2, about 50 mg of vitamin B5, about 50 mg of vitamin B6, about 1000 μg of vitamin B12, about 800 μg of folic acid, about 200 mg of magnesium glycinate, and about 100 μg of selenium.

### DETAILED DESCRIPTION OF THE INVENTION

The present invention provides multivitamin compositions comprising vitamin B1, vitamin B2, vitamin B5, vitamin B6, vitamin B12, folic acid, magnesium glycinate, and selenium. These compositions are effective and natural treatments for improving health in a mammal. In some embodiments, the multivitamin compositions are administered to a female mammal to improve neurologic and/or hormonal function in the female. Improved neurologic and/or hormone function includes, but is not limited to, improved mood, decreased usage of antidepressants, decreased usage of antianxiety medications, improved memory, increased energy, normalization of menstrual cycle, and increased length of sleep cycle.

The term “vitamin B1” includes thiamin hydrochloride, thiamin pyrophosphate (TPP), thiamin monophosphate (TMP), and thiamin diphosphate (TDP). In a preferred embodiment, the vitamin B1 is thiamin hydrochloride. The term “vitamin B5” includes pantothenol and calcium pantothenate. In a preferred embodiment, the vitamin B5 is calcium pantothenate. The term “vitamin B6” includes pyridoxine hydrochloride and pyridoxal 5′-phosphate (PLP). In a preferred embodiment, the vitamin B6 is pyridoxine hydrochloride. The term “vitamin B12” includes methylcobalamin, cyanocobalamin, hydroxycobalamin, and adenosylocobalamin. In a preferred embodiment, the vitamin B12 is methylcobalamin. The term “selenium” includes selenomethionine and sodium selenite. In a preferred embodiment, the selenium is selenomethionine.

In one embodiment of the present invention, the multivitamin composition comprises vitamin B1 in an amount of about 10 mg to about 40 mg. In another embodiment, the multivitamin composition comprises vitamin B2 in an amount of about 10 mg to about 40 mg. In another embodiment, the multivitamin composition comprises vitamin B5 in an amount of about 25 mg to about 75 mg. In another embodiment, the multivitamin composition comprises vitamin B6 in an amount of about 25 mg to about 75 mg. In another embodiment, the multivitamin composition comprises vitamin B12 in an amount of about 500 μg to about 1500 μg. In another embodiment, the multivitamin composition comprises folic acid in an amount of about 400 μg to about 1200 μg. In another embodiment, the multivitamin composition comprises magnesium glycinate in an amount of about 100 mg to about 300 mg. In another embodiment, the multivitamin composition comprises selenium in an amount of about 50 μg to about 150 μg. In a preferred embodiment, the multivitamin composition comprises a combination of vitamin B1, vitamin B2, vitamin B5, vitamin B6, vitamin B12, folic acid, magnesium glycinate, and selenium wherein the amounts of one or more of those components is as follows: about 10 mg to about 40 mg of vitamin B1, about 10 mg to about 40 mg of vitamin B2, about 25 mg to about 75 mg of vitamin B5, about 25 mg to about 75 mg of vitamin B6, about 500 μg to about 1500 μg of vitamin B12, about 400 μg to about 1200 μg of folic acid, about 100 mg to about 300 mg of magnesium glycinate, and about 50 μg to about 150 μg of selenium. As used herein, the term “multivitamin composition” includes one or more tablets or capsules as ingested daily such that the milligram amounts provided herein are daily intake amounts.

In other embodiments of the present invention, the multivitamin composition comprises about 25 mg of vitamin B1. In another embodiment, the multivitamin composition comprises about 25 mg of vitamin B2. In another embodiment, the multivitamin composition comprises about 50 mg of vitamin B5. In another embodiment, the multivitamin composition comprises about 50 mg of vitamin B6. In another embodiment, the multivitamin composition about 1000 μg of vitamin B12. In another embodiment, the multivitamin composition comprises about 800 μg of folic acid. In another embodiment, the multivitamin composition comprises about 200 mg of magnesium glycinate. In another embodiment, the multivitamin composition comprises about 100 μg of selenium. In a further preferred embodiment, the multivitamin composition comprises a combination of vitamin B1, vitamin B2, vitamin B5, vitamin B6, vitamin B12, folic acid, magnesium glycinate, and selenium wherein the amounts of one or more of those components is as follows: about 25 mg of vitamin B1, about 25 mg of vitamin B2, about 50 mg of vitamin B5, about 50 mg of vitamin B6, about 1000 μg of vitamin B12, about 800 μg of folic acid, about 200 mg of magnesium glycinate, and about 100 μg of selenium.

The multivitamin compositions of the present invention may be formulated in any of a variety of suitable forms, for example, oral, topical or parenteral administration. Standard pharmaceutical formulation techniques known by those of skill in the art may be used to prepare these formulations. Depending upon the particular route of administration, a variety of carriers well known in the art may be used. These include solid or liquid fillers, diluents, hydrotropes, surface-active agents and encapsulating substances. Optional pharmaceutically active or cosmetically active materials may be included which do not substantially interfere with the activity of the multivitamin compositions used in the methods of the present invention. The amount of carrier employed in conjunction with the multivitamin compositions used in the methods of the present invention is sufficient to provide a practical quantity of material for administration per unit dose of the compositions.

Typically, the multivitamin compositions of the present invention are administered orally. Various oral dosage forms can be used, including such solid forms as tablets, capsules, granules and bulk powders. In a preferred embodiment, a multivitamin composition is administered as a capsule. These oral forms comprise an effective amount of a multivitamin composition. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents.

Orally administered multivitamin compositions also include liquid solutions, emulsions, suspensions, powders, granules, elixirs, tinctures, syrups and the like. The carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel RC-591, tragacanth and sodium alginate; typical wetting agents include lecithin and polysorbate 80; and typical preservatives include methyl paraben and sodium benzoate. Peroral liquid multivitamin compositions may also contain one or more components such as sweeteners, flavoring agents or colorants as described above. Liquid oral dosage forms also include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

Other formulations useful for attaining systemic delivery of the multivitamin compositions of the present invention include sublingual, buccal and nasal dosage forms. Such multivitamin compositions typically comprise one or more soluble filler substances such as sucrose, sorbitol and mannitol; and binders such as acacia, microcrystalline cellulose, carboxymethyl cellulose and hydroxypropyl methylcellulose. Glidants, lubricants, sweeteners, colorants, antioxidants and flavoring agents described above may also be included.

The multivitamin compositions of the present invention can also be applied topically. The carrier of a topical multivitamin composition may aid penetration of the multivitamin composition into the skin. Such topical multivitamin compositions may be in any form including, for example, solutions, oils, creams, ointments, gels, lotions, pastes, shampoos, leave-on and rinse-out hair conditioners, milks, cleansers, moisturizers, sprays, aerosols, skin patches and the like. A variety of carrier materials well known in the art for topical application, such as, for example, water, alcohols, aloe vera gel, allantoin, glycerine, vitamin A and E oils, mineral oil, propylene glycol, and the like can be used to prepare such formulations.

Topical formulations are often prepared in the form of emulsions. The term “emulsion,” as used herein refers to mixtures of two or more liquids, which may be in the form of a continuous phase and a disperse phase, for example. Exemplary emulsions may be in the form of creams, lotions, ointments, gels, etc. and may include, for example, oil-in-water emulsions, water-in-oil emulsions, multiple emulsions and microemulsions. These formulations will be prepared which contain from about 0.001 to 10 w/w % of the multivitamin compositions of the present invention. These formulations will then be applied to the desired areas from 1 to 4 times daily. Alternatively, these formulations will be applied to the desired areas less frequently, i.e., from 1 to 5 times a week.

The multivitamin compositions may also be administered topically in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. A potential formulation for topical delivery of the multivitamin compositions used in the methods of the present invention utilizes liposomes such as described in U.S. Pat. No. 4,911,928 and U.S. Pat. No. 5,834,014.

Carriers for systemic administration include, for example, sugars, starches, cellulose and its derivatives, malt, gelatin, talc, calcium sulfate, vegetable oils, synthetic oils, polyols, alginic acid, phosphate buffer solutions, emulsifiers, isotonic saline and pyrogen-free water. Suitable carriers for parenteral administration include, for example, propylene glycol, ethyl oleate, pyrrolidone, ethanol, and sesame oil.

The formulations described above can be used in the methods of the present invention. The present invention includes methods of improving health in a mammal comprising administering a multivitamin composition to the mammal, wherein the composition comprises vitamin B1, vitamin B2, vitamin B5, vitamin B6, vitamin B12, folic acid, magnesium glycinate, and selenium. The term “improving health” refers herein to an increase in the mammal's sleep cycle length, an increase in the mammal's energy, a decrease in the mammal's need or use of an antidepressant, a decrease in the mammal's need or use of an antianxiety medication, normalization of the mammal's menstrual cycle, and/or an improvement of the mammal's hormonally based symptoms. Normalizing a mammal's menstrual cycle refers to returning a mammal's menstrual cycle to an approximately 28 day cycle. In some embodiments, normalizing a mammal's menstrual cycle further includes regular, monthly ovulation.

Also provided herein are methods of treating a sleep disorder comprising administering a multivitamin composition to the mammal, wherein the composition comprises vitamin B1, vitamin B2, vitamin B5, vitamin B6, vitamin B12, folic acid, magnesium glycinate, and selenium. The term “sleep disorder” includes, but is not limited to, insomnia, difficulty falling asleep, and difficulty staying asleep. In a preferred embodiment, the multivitamin composition is administered orally. In a further preferred embodiment, the composition is in the form of one or more capsules.

In one embodiment of the present invention, a multivitamin composition is administered to a mammal that comprises vitamin B1 in an amount of about 10 mg to about 40 mg. In another embodiment, a multivitamin composition is administered to a mammal that comprises vitamin B2 in an amount of about 10 mg to about 40 mg. In another embodiment, a multivitamin composition is administered to a mammal that comprises vitamin B5 in an amount of about 25 mg to about 75 mg. In another embodiment, a multivitamin composition is administered to a mammal that comprises vitamin B6 in an amount of about 25 mg to about 75 mg. In another embodiment, a multivitamin composition is administered to a mammal that comprises vitamin B12 in an amount of about 500 μg to about 1500 μg. In another embodiment, a multivitamin composition is administered to a mammal that comprises folic acid in an amount of about 400 μg to about 1200 μg. In another embodiment, a multivitamin composition is administered to a mammal that comprises magnesium glycinate in an amount of about 100 mg to about 300 mg. In another embodiment, a multivitamin composition is administered to a mammal that comprises selenium in an amount of about 50 μg to about 150 μg. In a preferred embodiment, a multivitamin composition is administered to a mammal that comprises a combination of vitamin B1, vitamin B2, vitamin B5, vitamin B6, vitamin B12, folic acid, magnesium glycinate, and selenium wherein the amounts of one or more of those components is as follows: about 10 mg to about 40 mg of vitamin B1, about 10 mg to about 40 mg of vitamin B2, about 25 mg to about 75 mg of vitamin B5, about 25 mg to about 75 mg of vitamin B6, about 500 μg to about 1500 μg of vitamin B12, about 400 μg to about 1200 μg of folic acid, about 100 mg to about 300 mg of magnesium glycinate, and about 50 μg to about 150 μg of selenium.

In other embodiments of the present invention, a multivitamin composition is administered to a mammal that comprises about 25 mg of vitamin B1. In another embodiment, a multivitamin composition is administered to a mammal that comprises about 25 mg of vitamin B2. In another embodiment, a multivitamin composition is administered to a mammal that comprises about 50 mg of vitamin B5. In another embodiment, a multivitamin composition is administered to a mammal that comprises about 50 mg of vitamin B6. In another embodiment, a multivitamin composition is administered to a mammal that comprises about 1000 μg of vitamin B12. In another embodiment, a multivitamin composition is administered to a mammal that comprises about 800 μg of folic acid. In another embodiment, a multivitamin composition is administered to a mammal that comprises about 200 mg of magnesium glycinate. In another embodiment, a multivitamin composition is administered to a mammal that comprises about 100 μg of selenium. In a further preferred embodiment, a multivitamin composition is administered to a mammal that comprises a combination of vitamin B1, vitamin B2, vitamin B5, vitamin B6, vitamin B12, folic acid, magnesium glycinate, and selenium wherein the amounts of one or more of those components is as follows: 25 mg of vitamin B1, 25 mg of vitamin B2, 50 mg of vitamin B5, 50 mg of vitamin B6, 1000 μg of vitamin B12, 800 μg of folic acid, 200 mg of magnesium glycinate, and 100 μg of selenium.

### Example

The compositions and methods provided herein will be further understood by reference to the following non-limiting example.

### Treatment of Patients with Multivitamin Compositions

A multitude of human patients were administered a composition comprising about 25 mg of vitamin B1, about 25 mg of vitamin B2, about 50 mg of vitamin B5, about 50 mg of vitamin B6, about 1000 μg of vitamin B12, about 800 μg of folic acid, about 200 mg of magnesium glycinate, and about 100 μg of selenium. The composition was administered daily (via self-administration) from one to three months with the following results.

Normalization of menstrual cycles—several patients experienced normalization of their menstrual cycle with three to four months of using the above-described multivitamin composition. Two patients menstrual cycles transitioned from 34 days and 17 days, respectively, to 28 days with predictable ovulation.

Increased energy—several patients presented with fatigue and an inability to function throughout the day due to tiredness. Following one month of treatment with the above-described multivitamin compositions, these patients fatigue decrease significantly.

Improved sleep—three patients presented with insomnia and difficulty falling asleep. Following one to two months of treatment with the above-described multivitamin compositions, each of these patients experienced improvement in sleep patterns with an increased ability to fall asleep.

It is understood that the examples and embodiments described herein are for illustrative purposes only and that various modifications or changes in light thereof will be suggested to person skilled in the art and are to be included with the spirit and purview of this applications and scope of the appended claims. All publications, patents and patent applications cited herein are hereby incorporated by reference in their entirety for all purposes.

What is claimed is:

1. A composition comprising about 25 mg of vitamin B1, about 25 mg of vitamin B2, about 50 mg of vitamin B5, about 50 mg of vitamin B6, about 1000 μg of vitamin B12, about 800 μg of folic acid, about 200 mg of magnesium glycinate, and about 100 μg of selenium.

2. The composition of claim 1, wherein the composition is a capsule.

3. The composition of claim 1, wherein the vitamin B1 is thiamin hydrochloride.

4. The composition of claim 1, wherein the vitamin B5 is calcium pantothenate.

5. The composition of claim 1, wherein the vitamin B6 is pyridoxine hydrochloride.

6. The composition of claim 1, wherein the vitamin B12 is a methylcobalamin.

7. The composition of claim 1, wherein the selenium is selenomethionine.

8. The composition of claim 1, wherein the vitamin B1 is thiamin hydrochloride, the vitamin B5 is calcium pantothenate, the vitamin B6 is pyridoxine hydrochloride, the vitamin B12 is a methylcobalamin, and the selenium is selenomethionine.

9. A method of normalizing a menstrual cycle of a mammal comprising administering the mammal a composition comprising about 25 mg of vitamin B1, about 25 mg of vitamin B2, about 50 mg of vitamin B5, about 50 mg of vitamin B6, about 1000 μg of vitamin B12, about 800 μg of folic acid, about 200 mg of magnesium glycinate, and about 100 μg of selenium.

10. The method of claim 9, wherein the mammal is a human.

11. The method of claim 9, wherein the composition is administered once daily.

12. The method of claim 9, wherein the vitamin B1 is thiamin hydrochloride, the vitamin B5 is calcium pantothenate, the vitamin B6 is pyridoxine hydrochloride, the vitamin B12 is a methylcobalamin, and the selenium is selenomethionine.

13. A method of treating a sleep disorder in a mammal comprising administering the mammal a composition comprising about 25 mg of vitamin B1, about 25 mg of vitamin B2, about 50 mg of vitamin B5, about 50 mg of vitamin B6, about 1000 μg of vitamin B12, about 800 μg of folic acid, about 200 mg of magnesium glycinate, and about 100 μg of selenium.

14. The method of claim 13, wherein the mammal is a human.

15. The method of claim 13, wherein the composition is administered once daily.

16. The method of claim 13, wherein the vitamin B1 is thiamin hydrochloride, the vitamin B5 is calcium pantothenate, the vitamin B6 is pyridoxine hydrochloride, the vitamin B12 is a methylcobalamin, and the selenium is selenomethionine.
